(19) United States
(12) Patent Application Publication
(10) Pub. No.: US 2016/0040235 A1
(43) Pub. Date: Feb. 11, 2016

(54) Methods for Diagnosing and Treating Diseases Caused by Genetic Copy Number Variants of Ultra-Conserved Elements

(71) Applicant: PRESIDENT AND FELLOWS OF HARVARD COLLEGE, Cambridge, MA (US)

(72) Inventors: Chao-ting Wu, Brookline, MA (US); Brian Beliveau, Cambridge, MA (US); Ruth Mccole, Brookline, MA (US)

(21) Appl. No.: 14/775,083

(22) PCT Filed: Mar. 11, 2014
(86) PCT No.: PCT/US2014/022922

§ 371 Date: Sep. 11, 2015

Related U.S. Application Data
(60) Provisional application No. 61/787,723, filed on Mar. 15, 2013.

(51) Int. Cl. C12Q 1/68

(52) U.S. Cl. CPC C12Q 1/6883; C12Q 2600/156

(57) ABSTRACT

Methods for inducing cell apoptosis by cellular comparison of genetic copy number variants of ultra-conserved elements.

### RELATED APPLICATION DATA

This application claims priority to U.S. Provisional Patent Application No. 61/787,723 filed on Mar. 15, 2013 and is hereby incorporated herein by reference in its entirety for all purposes.

### STATEMENT OF GOVERNMENT INTERESTS

This invention was made with Government support under grant number 1 R01 GM085169-01A1 awarded by NIH. The Government has certain rights in the invention.

### FIELD OF THE INVENTION

The present invention relates to ultra-conserved elements in a cell and the pairing of ultra-conserved elements in a cell as a method for treating an individual

### BACKGROUND OF THE INVENTION

Ultra-conserved elements are sequences that are perfectly conserved between reference genomes of distantly related species. Ultra-conserved elements (UCEs) have been reported, such as by Bejerano et al. who compared the reference genomes of human, mouse and rat to reveal an unexpected 481 orthologous genomic regions that are ≧200 bp in length and 100% identical in sequence. The reason why ultraconserved elements have been so extremely conserved for hundreds of millions of years is as of yet unexplained as neither enhancers, nor transcription factor binding sites, nor promoters, nor protein coding regions, nor any known function require such a high level of conservation (Bejerano et al., 2004; Fisher, Grice, Vinton, Bessling, & McCallion, 2006; Jaeger et al., 2010; Meireles-Filho & Stark, 2009; Taher et al., 2011; Visel et al., 2008; Weirauch & Hughes, 2010). However, UCEs have resisted sequence change for at least three to four hundred million years of evolution. Because roughly half of UCEs are intergenic and one quarter are intronic, a popular expectation is that UCEs will be found to embody regulatory activities, and, indeed, many are able to direct tissue-specific transcription (Lampe et al., 2008; Pennacchio et al., 2006; Poitras et al., 2010; Visel et al., 2008; Woolfe et al., 2005). However, the independent deletion of four noncoding UCEs from mice produced no obviously deleterious phenotypes (Ahituv et al., 2007). This finding suggested that although UCEs have remained essentially unchanged for millions of years, they are dispensable, at least for the four UCEs studied, under laboratory conditions. It also showed that UCEs cannot be assumed to have essential enhancer functions.

It is proposed that ultraconservation can be explained if the two copies of each UCE in a diploid cell, one on each of two homologous chromosomes, pair and then undergo sequence comparison (Derti, Roth, Church, & Wu, 2006) (Chiang et al., 2008; Kritsas et al., 2012; Vavouri & Lehner, 2009), wherein discrepancies in copy number or sequence of the UCEs being compared result in loss of fitness of the cell compared to the wild-type cell, leading eventually, in certain circumstances to cell death. Such a mechanism would enable the cell to sense and potentially respond to disruptions of genome integrity. Intriguingly, this model is consistent with the apparently normal phenotype of mice lacking a UCE on both homologous chromosomes (Ahituv et al., 2007), as homozygosity for the loss of a UCE would not lead to discrepancy in copy number or sequence. This model also predicts that UCEs are unlikely to be deleted or duplicated in the healthy genome. Indeed, significant depletion from segmental duplications (SDs) and copy number variants (CNVs) has been found (Chiang et al., 2008; Conrad et al., 2010; Derti et al., 2006; Kritsas et al., 2012), with depletion being driven primarily by the intronic and intergenic UCEs (Chiang et al., 2008; Derti et al., 2006).

Abnormal copy numbers of UCEs are associated with disease states (Chiang et al., 2008; Derti et al., 2006). Indeed, a positive association has been observed between ultraconserved regions and regions found to be deleted or duplicated in cancer (Calin et al., 2007). Several studies have also highlighted the possible roles for transcription of specific UCEs (known as Transcribed Ultraconserved Regions, T-UCRs) in cancer (Braconi et al., 2011; Lujambio et al., 2010; Mestdagh et al., 2010; Sana et al., 2012; Scaruffi et al., 2009). However, the cancer-associated copy number variant regions previously investigated (Calin et al., 2007) were not identified by comparing the genomes of cancer and healthy cells within the same individual, nor were they enriched for “driver” copy number aberrations, as is the current standard (Beroukhim et al., 2010). Accordingly, a need exists to understand UCEs in healthy and diseased cells and develop methods of diagnosis and treatment based on copy number variants of UCEs.

### SUMMARY

Embodiments of the present disclosure are directed to methods of inducing a cell to pair ultra-conserved elements (“UCEs”) such that if the cell has an abnormal UCE pairing, the cell will die. Alternate embodiments of the present disclosure are directed to methods of inducing a cell within a mammal to pair ultra-conserved elements (“UCEs”) on homologous chromosomes such that if the cell has an abnormal UCE pairing, the cell will die. In this manner, a therapeutic method is provided whereby cells including a copy number variation of one or more UCEs will be eliminated from the mammal, as a copy number variation of one or more UCEs may be indicative of a deleterious cell type.

According to one aspect, one or more cells being deficient in capability to compare UCEs on homologous chromosomes is induced to compare UCEs on homologous chromosomes, and those cells which include a copy number variation of one or more UCEs will apoptose.

Embodiments of the present disclosure are directed to a method of diagnosing an individual with a disease including the steps of obtaining a cell sample from the individual, comparing a maternal ultra-conserved element and a corresponding paternal ultra-conserved element, and diagnosing the individual with a disease when the maternal ultra-conserved element differs from the paternal ultra-conserved element.

However, aspects of the present disclosure do not require diagnosis where the method is directed to inducing cells, such as diseased cells, to pair homologous chromosomes and determine abnormal copy number counts of ultraconserved elements or failure of the cell to determine correct copy number counts of ultraconserved elements. The cell will then die and be eliminated from the populations of cells of which it was a member. According to one aspect, a cell having an abnormal copy number of UCEs or being unable to determine correct copy number counts of ultraconserved elements is a diseased cell and a population of cells benefits from the cell being removed therefrom. Accordingly, no diagnosis is require for therapeutic treatment of an individual to eliminate cells having an abnormal copy number of UCEs or being unable to determine correct copy number counts of ultraconserved elements using the methods described herein.

Embodiments of the present disclosure are further directed to a method of treating an individual for a disease related to copy number variation of an ultra-conserved element in one or more cells, including triggering or inducing recognition by the cell of the copy number variation of the ultra-conserved element leading to cell apoptosis or elimination from a population of cells. According to one aspect, the one or more cells are in a disease state. According to this aspect, cells are killed that are abnormal in UCE pairing.

Embodiments of the present disclosure are further directed to a method of treating an individual to eliminate cells having an abnormal copy number of UCEs or being unable to determine correct copy number counts of ultraconserved elements including triggering or inducing recognition by the cell of the copy number variation of the ultra-conserved element or the lack of the ability to determine correct copy number of UCEs thereby leading to cell apoptosis or elimination from a population of cells. According to one aspect, the one or more cells are in a disease state. According to this aspect, cells are killed that are abnormal in UCE pairing.

Embodiments of the present disclosure are further directed to a method of purging deleterious cells having copy number variation of an ultra-conserved element or the lack of the ability to determine correct copy number of UCEs from an individual comprising triggering recognition by the cell of the copy number variation of the ultra-conserved element leading to cell apoptosis or elimination from a population of cells.

Embodiments of the present disclosure are further directed to a method of purging a cell having copy number variation of an ultra-conserved element or the lack of the ability to determine correct copy number of UCEs from a population of cells comprising triggering recognition by the cell of the copy number variation of the ultra-conserved element leading to cell apoptosis, cell loss of fitness to survive or elimination from a population of cells.

Embodiments of the present disclosure are directed to a method of using ultra-conserved sequences to monitor and clear the genome of a population of cells from one or more cells having copy number variation of an ultra-conserved element or the lack of the ability to determine correct copy number of UCEs comprising triggering recognition by the cell of the copy number variation of the ultra-conserved element leading to cell apoptosis or elimination from a population of cells.

Embodiments of the present disclosure are directed to a method of eliminating cells from an individual comprising causing a cell to compare ultra-conserved elements from maternal DNA with ultra-conserved elements from paternal DNA, and wherein the cell becomes not viable if the ultra-conserved elements from the maternal DNA differ in sequence or copy number from the ultra-conserved elements from the paternal DNA or the cell lacks the ability to determine correct copy number of UCEs.

According to one aspect, cells described herein include pairing genes. According to this aspect, one or more pairing genes are activated by methods known to those of skill in the art, such as transfection, electroporation or transcriptional activation, to induce pairing of UCEs within a cell. If the pairing results in detection of a copy number variation of a UCE, then the cell will die.

According to one aspect, cells described herein include anti-pairing genes. According to this aspect, one or more anti-pairing genes are silenced by methods known to those of skill in the art, such as transfection, electroporation or transcriptional activation, to induce pairing of UCEs within a cell. If the pairing results in detection of a copy number variation of a UCE, then the cell will die.

According to one aspect, the one or more cells need not have copy number variations to result in an abnormal UCE pairing. Instead, the one or more cells may have a genetic rearrangement, such as an inversion or translocation, that prevents UCEs from pairing with each other in a normal manner to confirm identity. Such would be sufficient to trigger the one or more cells to die.

According to certain aspects, a method of making a population of cells having minimized copy number variants of ultra-conserved elements is provided including growing cells by doubling, and monitoring the cells for UCE copy number until copy number variants of UCEs are minimized According to one aspect, cells include any cell intended for placement within a mammal. The methods described herein reduce the likelihood that the cells will include copy number variants for UCEs which may lead to a deleterious cell type. According to certain aspects, the cells are doubled the following number of times: at least 1, 2, 3, 4, 5, 6, 7, 8, 9, 10, 15, 20, 25, 30 times and so on. Exemplary cells include iPS cells.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 illustrates the four types of copy number variation of the [resent disclosure. A: “classical” CNVs are genomic regions that vary in copy number between different individuals. B: ^{cancer}CNAs are copy number alterations that occur specifically in cancer cells, and that are absent in the healthy cells of the same individual. C: ^{somatic}CNVs are genomic regions that vary in copy number within the healthy somatic cells of an individual. D: ^{iPS}CNVs are regions of genomic copy number variation within a population of iPS cells, which are not variant in the fibroblast cells from which the iPS cells were derived.

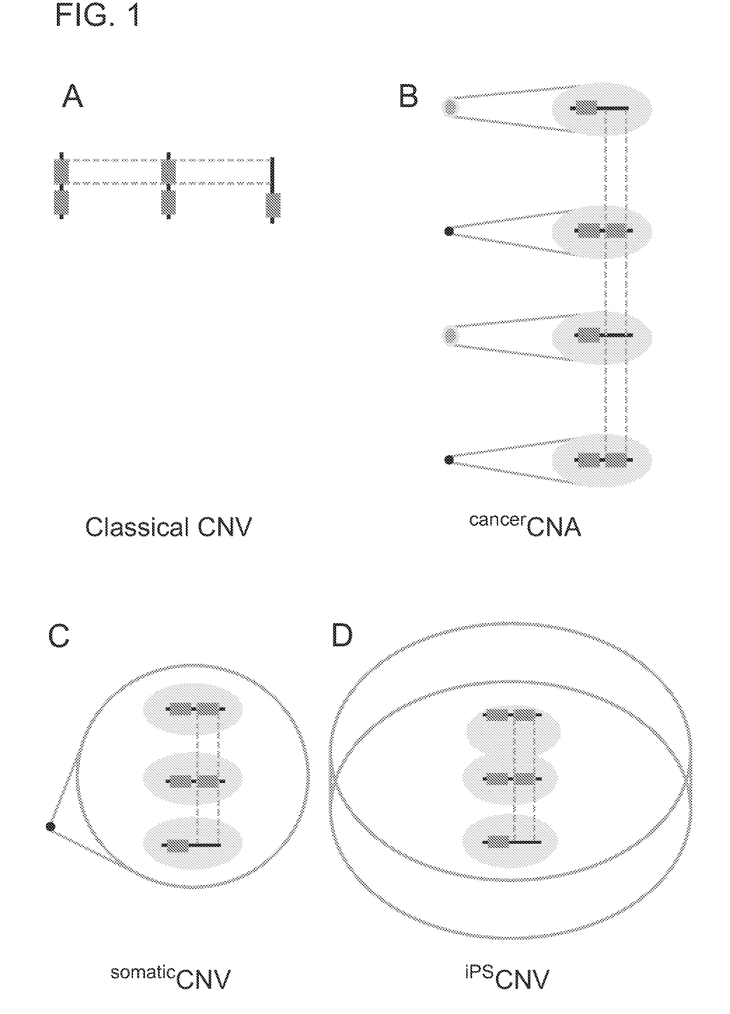

FIG. 2 is a heatmap showing correlation between the position of UCEs, “classical” CNVs and ^{cancer}CNAs, controlling for the genomic features listed on the x-axis, using partial correlations to “partition out” the correlation between UCEs and CNVs/CNAs that is attributable to the features listed, such as the position of genes, miRNAs, etc. The ^{cancer}CNAs row shows statistically significant positive correlation between the positions of UCEs and ^{cancer}CNAs. Likewise, the “classical” CNVs are negatively correlated with UCE position, as is expected since “classical” CNVs are depleted for UCEs. Importantly, all correlations remained statistically significant even when controlling for all the genomic features listed. Bins of 100 kb were used in this analysis. Bin sizes of 10 kb, 50 kb, 500 kb and 1 Mb produced similar results. All partial correlations between UCEs, “classical” CNVs and ^{cancer}CNAs were significant at all bin sizes. Color coding indicates direction (red for positive, green for negative) and strength of the partial correlation (brighter red for stronger positive correlation, brighter green for stronger negative correlation).

FIG. 3 depicts that UCEs are depleted from healthy CNVs even when the CNVs are less than a generation old, but enriched in ^{cancer}CNAs. Drawing together results from segmental duplications (Derti et al., 2006), the newest “classical” CNV datasets, ^{somatic}CNVs, ^{cancer}CNAs and CNVs from iPS cell culture, cells in culture may obtain a UCE-depleted CNV profile over time. Similarly, in healthy somatic human cells, a UCE-depleted profile of CNVs is seen, but that CNAs that arise specifically in cancer cells are enriched for UCEs.

### DETAILED DESCRIPTION

Aspects of the present disclosure are directed to methods of comparing ultra-conserved elements within a cell, for example, through pairing and comparison, and if the compared ultra-conserved elements differ, then the cell is culled from a population of cells and/or dies. Aspects of the present disclosure are directed to methods of comparing ultra-conserved elements within a cell, for example, through pairing and comparison by the cell, and if the compared ultra-conserved elements differ, then the cell is subject to apoptosis or is otherwise removed from tissue or cell clusters or the cell population at large.

According to certain aspects, certain genes involved in the pairing mechanism are disclosed in Joyce et al., PLoS Genetics, Vol. 8, Issue 5, e1002667 (2012) hereby incorporated by reference in its entirety.

UCEs are depleted from “classical” CNVs (Chiang et al., 2008; Derti et al., 2006). A UCE-depleted CNV profile requires a relatively long evolutionary timescale to be established, involving at least multiple human generations. According to the present disclosure, a UCE-depleted CNV profile is established in mitotically dividing cells, without germline transmission. According to this aspect, ^{somatic}CNAs (somatic copy number aberrations), established during the lifetime of an individual, without meiotic cell divisions or passage through the germline, are depleted for UCEs. According To additional aspects, methods are provided for determining UCE depletion from CNVs of diseased cells, such as cancer, which develop somatically within cell populations in the body.

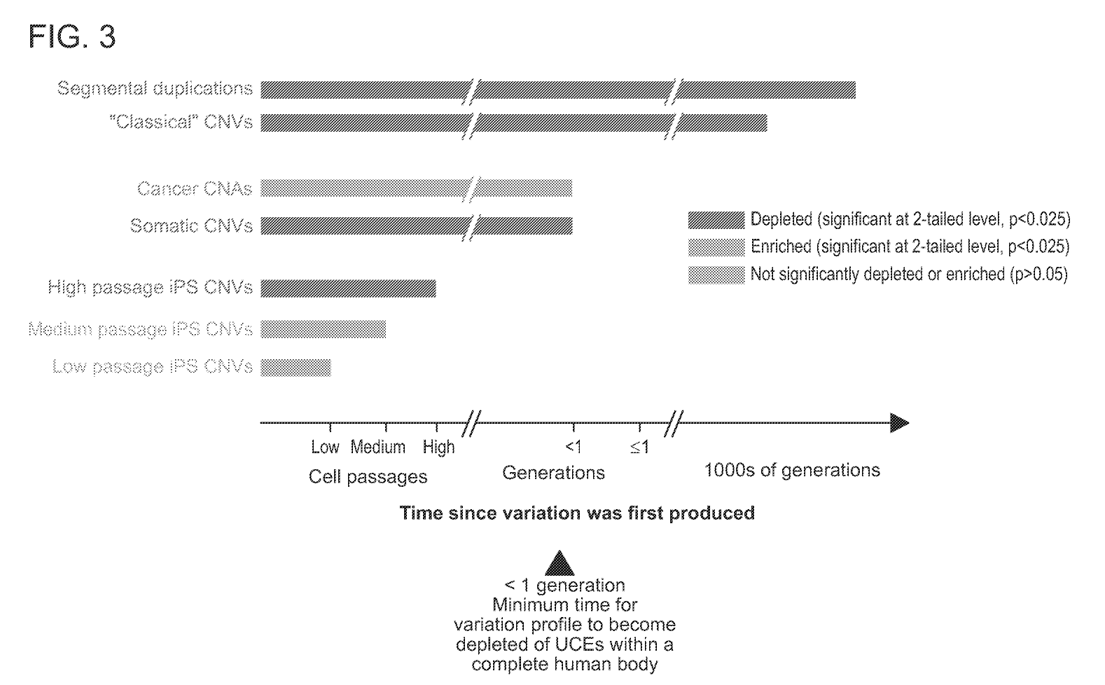

According to one aspect, a UCE-depleted CNV profile is generated in healthy cells, by studying CNVs over time in cell culture. As a result, human iPS cells move from an early state where they do not show a UCE-depleted profile of CNVs, to a later state, after more passages, where a UCE-depleted CNV profile is seen. According to one embodiment, healthy human cell populations purge themselves of copy number variant regions that overlap UCEs. Purging, such as rapid purging, can be accomplished by repair of CNVs disrupting UCEs, or removal of cells containing CNVs that disrupt UCEs, leaving behind a population of cells with a UCE-depleted CNV profile. According to an additional aspect, diseased cells, such as cancer cell populations, show above expected levels of UCE copy number disruption, evidenced by the enrichment of UCEs in ^{cancer}CNAs. See FIG. 3. Accordingly, methods of diagnosis or treatment are provided based on the contrast between ^{cancer}CNAs, enriched for UCEs, and ^{somatic}CNVs that are depleted for UCEs, including when ^{somatic}CNVs are called for individuals with cancer (Jacobs et al., 2012; Laurie et al., 2012). According to certain aspects, removed from analyses is any data where the tissue collected for ^{somatic}CNV calling was cancerous but data was used from patients who had cancer in a separate part of the body to where tissues were collected. Methods include identifying cells with enrichment of UCEs in ^{cancer}CNAs as an indication of the presence of a cancer progenitor cell likely to develop into cancer, as the enrichment of UCEs in ^{cancer}CNAs is an extremely cancer-specific phenomenon, because ^{somatic}CNVs from cancer patients are depleted for UCEs.

According to one aspect, healthy cells, such as iPS cells (induced pluripotent stem cells) containing a CNV that disrupts a UCE are disadvantaged, such as by having more rapid senescence, slower proliferation or a greater tendency to apoptose than cells without. In contrast, cancer cells with UCE-disrupting copy number variation are not similarly disadvantaged, but are at an advantage. This advantage could take the form of increased proliferative capacity, decreased propensity to apoptose, or other phenotypes.

According to one aspect, the dichotomy between somatic and cancer cells in the advantage of a UCE-enriched copy number variation profile arises from different subsets of UCEs being involved in the different effects seen in cancer cells and healthy cells. This would mean that the UCEs involved in the overall depletion of UCEs from ^{somatic}CNVs are not the same UCEs that are involved in the UCE enrichment in ^{cancer}CNAs. However, when considering the overlaps in UCEs with ^{cancer}CNAs and excluded from ^{somatic}CNVs, the largest group, comprising 312 UCEs, are both excluded from ^{somatic}CNVs but included within ^{cancer}CNAs. This suggests that in a healthy cell, the disruption of a set of UCEs by ^{somatic}CNVs is disadvantageous, whereas many of the same UCEs, when disrupted by ^{cancer}CNAs, provide an advantage to cancerous cells.

According to an additional aspect, healthy cells include a mechanism to translate the degree of UCE-CNV overlap within a cell into a competitive disadvantage in healthy cells, whereas in cancer cells this mechanism is absent. This absence of mechanism then allows UCE-CNV overlaps that are advantageous for the cancer cell to be established. For example, changing the copy number of certain transcribed UCEs may be advantageous to the cancer cell since some transcribed UCEs have been shown to act either as oncogenes (Braconi et al., 2011; Calin et al., 2007) or tumor suppressors (Lujambio et al., 2010). According to the present disclosure, one way in which UCE-CNV overlaps could be sensed by the cell and confer a selective disadvantage is if UCEs take part in a copy counting mechanism (Chiang et al., 2008; Derti et al., 2006; Kritsas et al., 2012; Vavouri & Lehner, 2009). If the maternal and paternal copies of a UCE were to recognize each other and compare their sequence, then a loss or duplication of a UCE because of an overlapping CNV could be detected, and induce deleterious mechanisms within the cell. Intriguingly, Michaelson et al (Michaelson et al., 2012) report that conserved sequences appear to occupy the more mutable parts of the human genome. However, mutations in conserved sequences do not tend to increase in frequency and become fixed in populations, since if they did the regions in question would not be considered conserved. According to one aspect, mutations introduced where a UCE has been disrupted by a CNV confer a strong selective disadvantage in healthy cells and do not endure in the human population.

Accordingly, methods are provided where cancer cells are induced to sense UCE-disrupting CNVs and then the cells become non-viable or otherwise are removed from cell populations.

The existence of opposite UCE-CNV profiles in healthy and cancerous human cell populations (UCEs depleted from CNVs in healthy cells, enriched in ^{cancer}CNAs in cancer cells, See FIG. 3) is the basis for methods of using the mechanism by which UCE-depleted CNV profiles are established in healthy cells to purge UCE-CNV overlaps in cancer cells. Because this would reduce the very high CNV burden present in these cells, and return the cells to a state more close to healthy cells, a method is provided to attenuate cancer cells and also to treat cancer.

This invention is further illustrated by the following examples, which should not be construed as limiting. The contents of all references, patents and published patent applications cited throughout this application are hereby incorporated by reference in their entirety for all purposes.

### Example I

### UCE Identification

The present Example is directed to whether the result that classical CNVs are depleted of UCEs (Chiang et al., 2008; Derti et al., 2006) is sensitive to the way UCEs were defined. To this end, two new UCE sets are defined, one using the dog, horse and cow reference genomes (builds used: canFam2, equCab2 and bosTau6), and one using mouse, rat and dog genomes (builds used mm9, rn4 and canFam2.) Pairwise alignments were found between each possible pair of genomes within the set of three, and elements with 100% basepair identity between each genome that were >200 bp in length were selected as the new sets of ultraconserved elements. These regions were then mapped to the hg18 human genome by BLAT (http://genome.ucsc.edu/cgi-bin/hgBlat), filtering out matches in the human genome that differed in length by more than 3 by and matches that were not unambiguously unique in the human genome. The hg18 orthologs of the new UCE sets were then used in the depletion analyses described herein. All recent high-quality CNV surveys were used (Campbell et al., 2011; Conrad et al., 2010; Iafrate et al., 2004; Jakobsson et al., 2008; Matsuzaki, Wang, Hu, Rava, & Fu, 2009; McCarroll et al., 2008; Shaikh et al., 2009), including personal CNV profiles determined by next-generation genome sequencing (1000 Genomes Project Consortium et al., 2010; Drmanac et al., 2010). The issue was whether the newly defined UCE sets are depleted from classical CNVs, as would be predicted if UCE depletion from CNVs is relatively insensitive to UCE definition. It was found that neither the use of alternative UCE definitions, nor new CNV datasets, altered in any way the depletion of UCEs from classical CNVs.

Accordingly, the result of classical CNVs being depleted of UCEs (Chiang et al., 2008; Derti et al., 2006) is not an artifact of a particular UCE definition. Remaining Examples use the UCE definition comprising all human-mouse-rat, human-dog-mouse and human-chicken UCEs used described in (Chiang et al., 2008; Derti et al., 2006).

### Example II

### Dataset Acquisition and Filtering

“Classical” CNV datasets: The coordinates of CNVs were obtained from the studies cited herein with the exception of Iafrate et al (Iafrate et al., 2004), which was obtained from the Database of Genetic Variants (http://projects.tcag.ca/variation/) as the Jan. 15, 2012 build. When necessary, coordinates were mapped to the hg18 genome build using the liftOver utility provided by UCSC (http://genome.ucsc.edu/cgi-bin/hgLiftOver). In each CNV database, overlapping regions were collapsed to avoid counting the same region multiple times. Unsequenced bases were excluded from all CNVs, leading to a final list of regions for each CNV data set that may differ from the original set reported in the relevant publication. Coordinates of CNVs from all datasets were combined to create a pooled somatic CNV dataset. Overlapping regions were merged, and CNVs from the Database of Genomic Variants (Iafrate et al., 2004) were excluded.

^{cancer}CNA datasets: ^{cancer}CNA datasets were obtained from (Beroukhim et al., 2010; Bullinger et al., 2010; Cancer Genome Atlas Network, 2012; Cancer Genome Atlas Research Network, 2011; Curtis et al., 2012; kConFab Investigators, Walker, Krause, Spurdle, & Waddell, 2012; Nik-Zainal et al., 2012; Taylor et al., 2010; The Cancer Genome Atlas Network et al., 2012; The Cancer Genome Atlas Research Network et al., 2012; Walter et al., 2009). Detailed information on the platforms used to detect ^{cancer}CNAs, the number of subjects, dataset coverage and ^{cancer}CNA size ranges were determined and provided in a data set. Following Beroukhim, Mermel et al (Beroukhim et al., 2010), all data were filtered to remove any ^{cancer}CNA longer than 50% of the length of the chromosome arm on which it resides. This is in order to remove ^{cancer}CNA calls that result from the loss of a whole chromosome or chromosome arm, which is considered a distinct type of genetic event from smaller deletions and duplications considered in the present disclosure.

Datasets were already filtered using an algorithm to contain ^{cancer}CNAs that are recurrent across samples and therefore more likely to be important for cancer causation or progression. These data sets were not filtered further for recurrent ^{cancer}CNAs. Three datasets; Bullinger et al (Bullinger et al., 2010), Walker et al (kConFab Investigators et al., 2012) and Nik-Zainal et al (Nik-Zainal et al., 2012) were not pre-filtered for recurrent variants and so a filter was performed whereby only ^{cancer}CNA regions that recurred (were present more than once in the dataset) were included. Of these two datasets, only data from Bullinger et al (Bullinger et al., 2010) and Nik-Zainal et al (Nik-Zainal et al., 2012) were included in the pooled ^{cancer}CNA dataset, because the recurrent ^{cancer}CNA regions from Walker et al (kConFab Investigators et al., 2012) covered 94% of the human genome and this was considered too large not to over-influence results from the pooled ^{cancer}CNA analyses. All pre-filtered datasets were included in the pooled ^{cancer}CNA analysis.

^{Somatic}CNV datasets: ^{somatic}CNVs were obtained from (Forsberg et al., 2012; O'Huallachain et al., 2012; Piotrowski et al., 2008) (Laurie et al., 2012) and (Jacobs et al., 2012). These datasets were filtered to remove any somaticCNA that is longer than 50% of the length of the chromosome arm on which it resides. All ^{somatic}CNV datasets were filtered to remove any ^{somatic}CNVs where the person in question had a cancer of the cell type from which the ^{somatic}CNV was called. This was in order not to confound the analysis of ^{somatic}CNVs by including regions that are not necessarily from healthy cells. For Jacobs et al (Jacobs et al., 2012), the excluded CNAs were those from patients with AML (Acute Myeloid Leukemia), CLL (Chronic Lymphocytic Leukemia), CML (Chronic Myelogenous Leukemia) and NHL (Non-Hodgkin Lymphoma) where blood samples were taken for ^{somatic}CNV discovery. For Laurie and Laurie et al (Laurie et al., 2012), excluded ^{somatic}CNVs are those in patients with ‘prior heamatological cancer’, where blood samples are used to discover ^{somatic}CNVs.

^{iPS}CNV datasets: Coordinates for ^{iPS}CNVs were obtained from Hussein et al (Hussein et al., 2011) in reference to the hg18 genome build. The study reported CNVs for multiple cell lines and at multiple passages; CNVs were stratified by their passage into low, medium and high passage CNVs. As the parental fibroblast strains were genotyped, CNV regions were removed that overlapped CNVs found in the fibroblast cells used to produce the iPS cells.

microRNAs: Human microRNA genomic positions were obtained with respect to genome build hg19 from ftp://mirbase.org/pub/mirbase/CURRENT/genomes/hsa.gff3. They were converted to hg18 using UCSC's liftover feature (http://genome.ucsc.edu/cgi-bin/hgLiftOver). For all analyses, the genomic positions of the microRNA precursor sequences, which are larger in by than the genomic regions that produce the processed microRNAs, were used.

### Example III

### Determining Depletion from or Enrichment of UCEs in Genomic Regions of Interest

Tests for depletion or enrichment of UCEs from various genomic regions such as CNV datasets were conducted as previously described (Chiang et al., 2008; Derti et al., 2006). Briefly, sets of genomic regions matched in number and length to each UCE were selected from any random position within the genome, excluding unsequenced regions. The base pair overlap between this set of random elements and the genomic feature in question was calculated. The process of creating a random set and calculating the base pair overlap was repeated 1000 times, creating an expected distribution. The observed result was compared to the expected distribution, and a P-value was calculated using a Z-test to determine the statistical significance of any depletion or enrichment. Two-tailed tests were performed with p-value cutoff for statistically significant depletion at 0.025 and statistically significant enrichment at 0.975. As this test is dependent on the underlying expected distribution approximating a normal distribution, the expected distribution for deviations from normality was tested using both Q-Q plots and the Kolmogorov-Smirnov test. These tests were performed for each pooled CNV dataset (“classical” CNVs, ^{cancer}CNAs, ^{somatic}CNVs and, iPS CNVs).

Copy number changes in cancer cells are enriched for UCEs. UCEs in healthy cells are maintained in correct copy number by avoiding CNVs. Disruption of UCE copy number by CNVs is associated with diseases such as cancer (Derti et al., 2006). According to the present disclosure, ^{cancer}CNAs, identified as specific to cancer cells and enriched for cancer “driver” events, are depleted of UCEs.

To ensure that ^{cancer}CNA data is of the highest quality and enriched in important “driver” aberrations, all studies included met certain standards. ^{cancer}CNAs only come from studies where cancer genomes were compared with healthy genomes from the same patients. This ensures that “classical” CNVs are not inadvertently included in the ^{cancer}CNA dataset, and that all aberrations are specific to cancer cells. Additionally, recurrent aberrations are considered more likely to be causal “drivers”, whilst non-recurrent ones are more likely to be non-functional “passengers”. Only recurrent aberrations were allowed into the ^{cancer}CNA set, identified as such using the tools GISITC (Mermel et al., 2011), RAE (Taylor et al., 2008), and analysis of recurrence. One study from which data for ^{cancer}CNA set was drawn also included a separate dataset where classical CNVs were identified in the same cancer patients (Curtis et al., 2012). Almost statistically significant depletion of UCEs from classical CNVs in cancer patients was observed, confirming the data quality for ^{cancer}CNAs. ^{cancer}CNAs are not depleted for UCEs. Indeed, they are significantly enriched for UCEs.

This means that copy number aberrations, specific to cancer cells, disrupt UCE copy number significantly more than would be expected by chance. The enrichment is not an artifact of the large size of ^{cancer}CNA regions, nor their overall coverage, nor of the tendancy of UCEs to cluster. Possible biological explanations for the enrichment of UCEs in ^{cancer}CNAs was explored.

### Example IV

### Controls for UCE Location Relative to Genes

Additional analyses were conducted in which UCEs were segregated into exonic, intronic and intergenic categories depending on their overlap with exons or introns. In these tests, random elements were drawn solely from the exonic, intronic or intergenic portions of the genome. Additionally, the possibility that the tendency of UCEs to appear in clusters could bias our analyses was considered. Thus, UCEs were joined that lay within a certain distance of a neighboring UCE using distance criteria increasing in size from 10 kb to 1 Mb, retaining the distance between these elements when selecting matching random elements. The positions of these random elements within the clusters were randomly permuted 1000 times, and the overlap calculated for each permutation.

UCEs within or near genes do not drive UCE enrichment in ^{cancer}CNAs. It was investigated whether the proximity of UCEs to genes could explain the enrichment of UCEs within ^{cancer}CNAs. The top 20 genes containing the most UCEs that also fall within ^{cancer}CNAs were identified. Upon removal of the 131 UCEs within these genes from analysis, enrichment of the remaining 765 UCEs in ^{cancer}CNAs was maintained (p=0.985, obs/exp=1.074). When only UCEs that do not occur within genes are examined, these UCEs are still enriched in ^{cancer}CNAs (p=0.991, obs/exp=1.129). UCEs in close proximity to genes are believed to be have a role in the enrichment of UCEs in ^{cancer}CNAs. All UCEs from analysis that are in genes or within 10 kb, 50 kb or 100 kb of any gene were removed. Enrichment was only lost when all UCEs within 100 kb of genes were removed from the analysis, leaving only 149 UCEs from a starting total of 896 (for 10 kb, p=0.991, obs/exp=1.129, for 50 kb, p=0.992, obs/exp=1.150, for 100 kb, p=0.952, obs/exp=1.127).

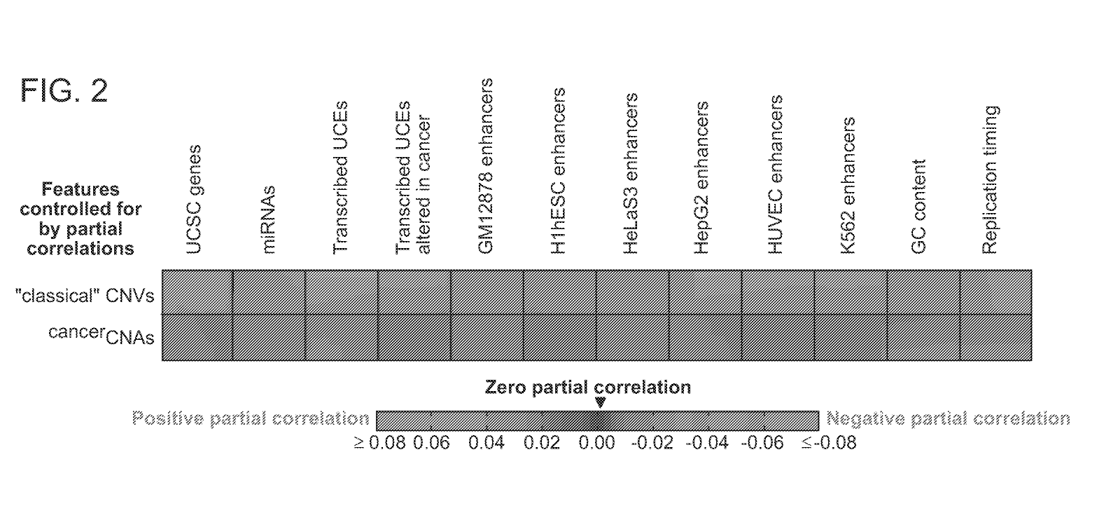

Partial correlation analysis was also used to address the question of whether the position of genes relative to UCEs explains the enrichment of UCEs in ^{cancer}CNAs. The positions of UCEs and ^{cancer}CNAs were correlated, and partial correlation was used to statistically remove the correlation between UCEs and ^{cancer}CNAs which is due to the positions of genes. The remaining partial correlation coefficient describes the level of correlation between UCEs and ^{cancer}CNAs that is independent of the location of all genes in the genome and was significant (p<0.05, See FIG. 2).

### Example V

### Controls for the Propensity for UCEs to Cluster

The possibility that the tendency of UCEs to appear in clusters could bias the analyses was considered. UCEs were joined that lay within a certain distance of a neighboring UCE using distance criteria increasing in size from 10 kb to 1 Mb, and retaining the distance between these elements when selecting matching random elements. The positions of these random elements within the clusters were randomly permuted 1000 times, and the overlap calculated for each permutation.

### Example VI

### Partial Correlations

Data for genomic features of interest was obtained from the following sources: UCSC genes: UCSC known genes track, build hg18; Enhancer regions: ENCODE Genome segmentation combined segmentation from the ENCODE UCSC hub (ENCODE Project Consortium et al., 2012), ‘E’ (enhancer) class genomic regions, enhancer regions for six ENCODE cell/tissue types are included; miRNAs: miRBase (Kozomara & Griffiths-Jones, 2011); GC content: UCSC genome browser.

Analyses were performed over 10 kb, 50 kb, 100 kb, 500 kb and 1 Mb bins. Results were similar for all bin sizes with no changes in significance for “classical” CNVs or ^{cancer}CNAs. Positional data was converted to a density measurement using a python script by summing the number of bases in a 100 kb window covered by the feature of interest (e.g. UCE, CNV), divided by the number of sequenced bases in the hg18 human genome within the same window.

Partial correlations were performed using Matlab partialcorr function.

### Example VII

### MicroRNA and Transcribed UCEs Studies

MicroRNAs are enriched in ^{cancer}CNAs but do not account for the enrichment of UCEs in ^{cancer}CNAs. MicroRNAs have previously been shown to be associated with regions of the genome that are fragile, and also with regions shown to be copy number variant in cancer cells (Calin et al., 2007; 2004). If microRNAs and UCEs have shown a similar relationship to regions of importance in cancer (Calin et al., 2004; 2007), then UCE results may merely mirror an effect that is actually centered on microRNAs. It was therefore examined whether miRNAs are enriched in ^{cancer}CNAs by treating miRNAs as though they were UCEs and running analyses exactly as before. miRNAs were found to be enriched in ^{cancer}CNAs, in line with previous results (Calin et al., 2007), p>0.999, obs/exp=1.101. Using partial correlations, the correlation between ^{cancer}CNAs and UCEs which is independent of the positions of miRNAs was partitioned out (See FIG. 2). A statistically significant positive partial correlation remains between UCEs and ^{cancer}CNAs, regardless of any relationship between miRNAs and ^{cancer}CNAs, demonstrating that the enrichment of miRNAs in ^{cancer}CNAs does not explain the enrichment of UCEs in ^{cancer}CNAs.

Transcribed UCEs contribute to, but are not fully responsible for, enrichment of UCEs in ^{cancer}CNAs. It was determined whether T-UCRs are specifically enriched within ^{cancer}CNAs. T-UCRs as a whole class are not significantly enriched in ^{cancer}CNAs (p=0.789, obs/exp=1.053). Examined just those T-UCRs whose expression level differs between cancer (chronic lymphocytic leukemia, colorectal cancer, hepatocellular carcinomas) and control tissues, It was found that though these UCEs are not significantly enriched in ^{cancer}CNAs, the p-value was close to significance (p=0.944, obs/exp=1.173), which suggests that these T-UCRs may contribute to the enrichment of UCEs as a whole group in ^{cancer}CNA regions, but do not drive the enrichment. This result is supported by partial correlation analysis that shows a statistically significant positive correlation between the position of UCEs and ^{cancer}CNAs, even when correlation between T-UCRs or T-UCRs with altered expression in cancer is statistically removed (See FIG. 2). Even when correlation between ^{cancer}CNA and UCE attributable both to miRNA and T-UCR positions was removed, the partial correlation between ^{cancer}CNAs and UCEs remained significant (p<0.05), showing that even the positions of T-UCRs and miRNAs combined does not explain the enrichment of UCEs in ^{cancer}CNAs.

UCEs are enriched within ^{cancer}CNA regions. Because these regions likely represent “driver” mutations or at least recurrently aberrant regions of the genome in cancer, UCEs occupy a fundamental role in cancer causation or progression. This effect is not due to a correlation of UCE positioning with genes, miRNAs, or enhancer regions, and UCE enrichment within ^{cancer}CNA regions is not affected by variation in GC content or replication timing across the genome. The partial correlations between classical CNV and UCE position, controlling for the same features as for ^{cancer}CNAs are all negative and statistically significant (See FIG. 2).

### Example VIII

### Very Young, Somatic CNAs are Depleted for UCEs

The present Example is directed to the timing of UCE depletion observed in classical CNVs. Datasets detailing somatic CNVs arising in non-cancerous cells were analyzed (^{somatic}CNVs). (Bruder et al., 2008; Forsberg et al., 2012; Jacobs et al., 2012; Laurie et al., 2012; O'Huallachain, Karczewski, Weissman, Urban, & Snyder, 2012; Piotrowski et al., 2008). Many individuals in these studies were cancer patients. To minimize the effect of this on ^{somatic}CNVs, all individuals were removed from consideration where the cancer-affected tissue and the tissue used to call somatic CNVs coincided (e.g. a person with leukemia where blood was sampled to discover somatic CNVs.) Using this filtered data, it was examined whether ^{somatic}CNVs are depleted for UCEs. Somatic CNAs are significantly depleted for UCEs (p=0.012, obs/exp=0.904). Any set of somatic copy number variant regions, whether from a healthy cell or a cancer cell, is not enriched for UCEs, purely because of the youth of the CNVs in question. The enrichment of UCEs in ^{cancer}CNAs is specific to the disease state. The depletion of UCEs from ^{somatic}CNVs also indicates that within a human's lifetime, the depletion of UCEs from CNVs that we first observed with older, “classical” CNVs is already being established.

### Example IX

### iPS Cell Lines “Purge” UCE-Overlapping CNVs to Obtain a UCE-Depleted CNV Profile

Even in populations of CNVs<1 generation old, a UCE-depleted profile of CNV positions is seen. One explanation for this is that when ^{somatic}CNVs disrupt the dosage of UCEs, this induces a fitness cost for the cell. The effect of this process on a population of cells would be that the profile of ^{somatic}CNVs is depleted for UCEs.

CNV profiles are measured over time in cellular populations. Data in iPS cells generated by Hussein et al (Hussein et al., 2011) for a different analysis was suitable for this analysis. Hussein et al. used the Affymmetrix SNP 6.0 microarray to characterize CNVs in 22 human iPS cell lines, as well as the three “parental” fibroblast lines from which iPS cells were generated.

CNVs in iPS cells were investigated that were not detected in the primary fibroblast cells used to make iPS cells. These are referred to as ^{iPS}CNVs. These CNVs could have arisen from two sources: either they occurred de novo as a result of the iPS cell formation protocol (Hussein et al., 2011; Laurent et al., 2011; Mayshar et al., 2010; Quinlan et al., 2011), or they were present in the fibroblast cells from which the iPS cells were made, but at levels below the limit of detection (Abyzov et al., 2012). For purposes of this Example, the two sources of ^{iPS}CNVs were not differentiated.

^{iPS}CNVs overlap UCEs as much as expected by chance (p=0.449, obs/exp=0.961). In other words, UCEs were not depleted from these early passage ^{iPS} CNVs. When iPS cells of a medium number of passages were examined again these ^{iPS}CNVs were not depleted for UCEs, although the p-value was closer to significance (p=0.068, obs/exp=0.662).

When late passage iPS cells (passage 12 to 26) were considered, a UCE-depleted ^{iPS}CNVs profile was seen (p=0.010, obs/exp=0.110). This result shows that although the profile of ^{iPS}CNVs begun as non-depleted for UCEs, over time it became depleted for UCEs.

According to the present disclosure, “classical” CNVs are depleted for UCEs, even with alterations to UCE definition. The property of UCE depletion from CNV regions is evolutionary conserved between many different mammals and is therefore evolutionarily old. In contrast to this, UCEs are enriched for ^{cancer}CNAs that have appeared over and over again in separate cancer samples, and may even include “driver” aberrations that underlie the progression of a cell into a cancerous state. This was not a function of the ^{cancer}CNAs being relatively young, because somatic CNAs<1 generation old, are depleted for UCEs. Finally, iPS cells are able to develop a UCE-depleted ^{iPS}CNVs profile in culture, providing an assay to study the establishment of a UCE-depleted CNV profile.

### Example X

### Targeting Ultra-Conserved Elements Using FISH

Amplification free probes are used to identify UCEs in cells using FISH-based methods. Accordingly, a method is provided whereby genomic DNA, such as DNA in a chromosome, is labeled and visualized without amplification of the hybridization probes. Other useful probes include a common binding site shared by a set of probes for binding of a common moiety bearing a label, such as with secondary labeling. For example, the common binding site may be a common nucleic acid sequence shared by the probe set. A labeled complementary sequence is then used to hybridize to the common nucleic acid sequence. In this manner, all probes within the set may be commonly labeled in an easy and efficient manner. This secondary labeling strategy is referred to as “mainstreet.”

According to one aspect of mainstreet, the use of a common binding site shared among the set of probes for a secondary label can turn a region targeted by a set of probes with unique genomic sequences into a “repeat” region where there is a high local concentration of binding sites for binding to a secondary label, i.e. a common secondary label. Regions of highly repeated sequences are appropriate targets for the methods describe herein. Since the labeling is secondary, large quantities of probes can be made and hybridized without the need for amplification. The large number of hybridized probes can then be similarly labeled using a secondary label common to all of the probes. Such large quantities of probes that can be secondarily labeled by a common secondary label enable whole-genome RNAi screens. Accordingly, a method is provided including hybridization of a mixture of nucleic acid probes bearing a common binding site to a target nucleic acid, such as DNA of a chromosome, nonchromosomal DNA, RNA, etc., binding a common secondary label to the hybridized nucleic acid probes and detection of the hybridized labeled probes which are sufficient in number to generate a detectable signal. According to one aspect, the probes are made without an amplification process. The large number of probes in a given probe set, such as about 300-400 probes, enables sufficient signal for detection.

According to one aspect, the probes having a common binding site for a secondary label are targeted to regions that are not frequently copy number variable such that the number of FISH signals will be a reliable proxy for the number of chromosomes. Exemplary targeted regions includes UCEs and other very highly conserved sequences which may be about 95%, 96%, 97%, 98% or 99% identical. UCEs are useful to mark dosage-sensitive regions of the genome i.e., regions that duplication and deletion of are not easily tolerated. Accordingly, UCEs are useful to enumerate chromosome number and are also useful as a control in an assay for counting the copy number of chromosomes or subchromosomal regions. UCEs are discussed in Bejerano et al., Science 304: 1321-25 (2004) hereby incorporated by reference.

Exemplary FISH methods include standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides. The reagents used in each of these steps and their conditions of use vary depending on the particular situation and whether their use is required with any particular probes. Hybridization conditions are also described in U.S. Pat. No. 5,447,841. It will be appreciated that numerous variations of in situ hybridization protocols and conditions are known and may be used in conjunction with the present invention by practitioners following the guidance provided herein.

Oligonucleotide probes useful for labeled probes according to the present disclosure may have any desired nucleotide length and nucleic acid sequence. Accordingly, aspects of the present disclosure are directed to the use of a plurality or set of nucleic acid probes, such as single stranded nucleic acid probes, such as oligonucleotide paints. Additional labeled probes include those known as “oligopaints” as described in US 2010/0304994. The term “probe” refers to a single-stranded oligonucleotide sequence that will recognize and form a hydrogen-bonded duplex with a complementary sequence in a target nucleic acid sequence or its cDNA derivative. The probe includes a target hybridizing nucleic acid sequence. Exemplary nucleic acid sequences may be short nucleic acids or long nucleic acids. Exemplary nucleic acid sequences include oligonucleotide paints. Exemplary nucleic acid sequences are those having between about 1 nucleotide to about 100,000 nucleotides, between about 3 nucleotides to about 50,000 nucleotides, between about 5 nucleotides to about 10,000 nucleotides, between about 10 nucleotides to about 10,000 nucleotides, between about 10 nucleotides to about 1,000 nucleotides, between about 10 nucleotides to about 500 nucleotide, between about 10 nucleotides to about 100 nucleotides, between about 10 nucleotides to about 70 nucleotides, between about 15 nucleotides to about 50 nucleotides, between about 20 nucleotides to about 60 nucleotides, between about 50 nucleotides to about 500 nucleotides, between about 70 nucleotides to about 300 nucleotides, between about 100 nucleotides to about 200 nucleotides, and all ranges or values in between whether overlapping or not. Exemplary oligonucleotide probes include between about 10 nucleotides to about 100 nucleotides, between about 10 nucleotides to about 70 nucleotides, between about 15 nucleotides to about 50 nucleotides, between about 20 nucleotides to about 60 nucleotides and all ranges and values in between whether overlapping or not. According to one aspect, oligonucleotide probes according to the present disclosure should be capable of hybridizing to a target nucleic acid. Probes according to the present disclosure may include a label or detectable moiety as described herein. Oligonucleotides or polynucleotides may be designed, if desired, with the aid of a computer program such as, for example, DNAWorks, or Gene2Oligo.

According to certain aspects, nucleic acid probes may include a primary nucleic acid sequence that is non-hybridizable to a target nucleic acid sequence in addition to the sequence of the probe that hybridizes to the target nucleic acid sequence. Exemplary primary nucleic acid sequences or target non-hybridizing nucleic acid sequences include between about 10 nucleotides to about 100 nucleotides, between about 10 nucleotides to about 70 nucleotides, between about 15 nucleotides to about 50 nucleotides, between about 20 nucleotides to about 60 nucleotides and all ranges and values in between whether overlapping or not. According to certain aspects, the primary nucleic acid sequence is hybridizable with one or more secondary nucleic acid sequences. According to certain aspects, the secondary nucleic acid sequence may include a label. According to this aspect, the nucleic acid probes are indirectly labeled as the secondary nucleic acid binds to the primary nucleic acid thereby indirectly labeling the probe which hybridizes to the target nucleic acid sequence. According to certain aspects, a plurality of nucleic acid probes is provided with each having a common primary nucleic acid sequence. That is, the primary nucleic acid sequence is common to a plurality of nucleic acid probes, such that each nucleic acid probe in the plurality has the same or substantially similar primary nucleic acid sequence. According to one aspect, the primary nucleic acid sequence is a single sequence species. In this manner, a plurality of common secondary nucleic acid sequences is provided which hybridize to the plurality of common primary nucleic acid sequences. That is, each secondary nucleic acid sequence has the same or substantially similar nucleic acid sequence. According to one exemplary embodiment, a single primary nucleic acid sequence is provided for each of the nucleic acid probes in the plurality. Accordingly, only a single secondary nucleic acid sequence which is hybridizable to the primary nucleic acid sequence need be provided to label each of the nucleic acid probes. According to certain aspects, the common secondary nucleic acid sequences may include a common label. According to this aspect, a plurality of nucleic acid probes are provided having substantially diverse nucleic acid sequences hybridizable to different target nucleic acid sequences and where the plurality of nucleic acid probes have common primary nucleic acid sequences. Accordingly, a common secondary nucleic acid sequence having a label may be used to indirectly label each of the plurality of nucleic acid probes. According to this aspect, a single or common primary nucleic acid sequence and secondary nucleic acid sequence pair can be used to indirectly label diverse nucleic acid probe sequences. Such an embodiment is provided where a plurality of nucleic acid probes having primary nucleic acid sequences are commercially synthesized, such as on an array. Labeled secondary nucleic acid sequences can also be commercially synthesized so that they are hybridizable with the primary nucleic acid sequences. The nucleic acid probes may be combined with the labeled secondary nucleic acids and one or more or a plurality of target nucleic acid sequences under conditions such that the nucleic acid probe or probes hybridize to the target nucleic acid sequence or sequences while the primary nucleic acid sequence is nonhybridizable to the target nucleic acid sequence or sequences. A labeled secondary nucleic acid sequence hybridizes with a corresponding primary nucleic acid sequence to indirectly label the nucleic acid probe, thereby labeling the target nucleic acid sequence. According to one aspect, the nucleic acid probes may be combined with the labeled secondary nucleic acids and one or more or a plurality of target nucleic acid sequences together in a one pot method. According to one aspect, the nucleic acid probes may be combined with the labeled secondary nucleic acids and one or more or a plurality of target nucleic acid sequences sequentially, such as the nucleic acid probes are combined with the target nucleic acid to form a mixture and then the labeled secondary nucleic acid is combined with the mixture or the nucleic acid probes are combined with the labeled secondary nucleic acids to form a mixture and then the target nucleic acid is combined with the mixture.

According to certain aspects, the primary nucleic acid sequence is modifiable with one or more labels. According to this aspect, one or more labels may be added to the primary nucleic acid sequence using methods known to those of skill in the art.

According to an additional embodiment, nucleic acid probes may include a first half of a ligand-ligand binding pair, such as biotin-avidin. Such nucleic acid probes may or may not include a primary nucleic acid sequence. The first half of a ligand-ligand binding pair may be attached directly to the nucleic acid probe. According to certain aspects, a second half of the ligand-ligand binding pair may include a label. Accordingly, the nucleic acid probe may be indirectly labeled by the use of a ligand-ligand binding pair. According to certain aspects, a common ligand-ligand binding pair may be used with a plurality of nucleic acid probes of different nucleic acid sequences. Accordingly, a single species of ligand-ligand binding pair may be used to indirectly label a plurality of different nucleic acid probe sequences. The common ligand-ligand binding pair may include a common label or a plurality of common ligand-ligand binding pairs may be labeled with different labels. Accordingly, a plurality of nucleic acid probes of different nucleic acid sequences may be labeled with a single species of label using a single species of a ligand-ligand binding pair.

According to one aspect, the primary nucleic acid sequences may include one or more subsequences that are hybridizable with one or more different secondary nucleic sequences. The one or more secondary nucleic acid sequences may include one or more subsequences that hybridize with one or more tertiary nucleic acid sequences, and so on. Each of the primary nucleic acid sequences, the secondary nucleic acid sequences, the tertiary nucleic acid sequences and so on may be directly labeled with a label or may be indirectly labeled with a label. In this manner, an exponential labeling of the nucleic acid probe can be achieved.

### Labels

A label according to the present disclosure includes a functional moiety directly or indirectly attached or conjugated to a nucleic acid which provides a desired function. According to certain aspects, a label may be used for detection. Detectable labels or moieties are known to those of skill in the art. According to certain aspects, a label may be used to retrieve a particular molecule. Retrievable labels or moieties are known to those of skill in the art. According to certain aspects, a label may be used to target a particular molecule to a target nucleic acid of interest for a desired function. Targeting labels or moieties are known to those of skill in the art. According to certain aspects, a label may be used to react with a target nucleic acid of interest. Reactive labels or moieties are known to those of skill in the art. According to certain aspects, a label may be an antibody, ligand, hapten, radioisotope, therapeutic agent and the like.

As used herein, the term “retrievable moiety” refers to a moiety that is present in or attached to a polynucleotide that can be used to retrieve a desired molecule or factors bound to a desired molecule (e.g., one or more factors bound to a targeting moiety). As used herein, the term “retrievable label” refers to a label that is attached to a polynucleotide (e.g., an Oligopaint) and can, optionally, be used to specifically and/or nonspecifically bind a target protein, peptide, DNA sequence, RNA sequence, carbohydrate or the like at or near the nucleotide sequence to which one or more Oligopaints have hybridized. In certain aspects, target proteins include, but are not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

As used herein, the term “targeting moiety” refers to a moiety that is present in or attached to a polynucleotide that can be used to specifically and/or nonspecifically bind one or more factors that associate with, modify or otherwise interact with a nucleic acid sequence of interest (e.g., DNA (e.g., nuclear, mitochondrial, transfected and the like) and/or RNA), including, but not limited to, a protein, a peptide, a DNA sequence, an RNA sequence, a carbohydrate, a lipid, a chemical moiety or the like at or near the nucleotide sequence of interest to which the polynucleotide has hybridized. In certain aspects, factors that associate with a nucleic acid sequence of interest include, but are not limited to histone proteins (e.g., H1, H2A, H2B, H3, H4 and the like, including monomers and oligomers (e.g., dimers, tetramers, octamers and the like)) scaffold proteins, transcription factors, DNA binding proteins, DNA repair factors, DNA modification proteins (e.g., acetylases, methylases and the like).

In other aspects, factors that associate with, modify or otherwise interact with a nucleic acid sequence of interest are proteins including, but not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) acetylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

In certain aspects, a targeting and/or retrievable moiety is activatable. As used herein, the term “activatable” refers to a targeting and/or retrievable moiety that is inert (i.e., does not bind a target) until activated (e.g., by exposure of the activatable, targeting and/or retrievable moiety to light, heat, one or more chemical compounds or the like). In other aspects, a targeting and/or retrievable moiety can bind one or more targets without the need for activation of the targeting and/or retrievable moiety. Exemplary methods for attaching proteins, lipids, carbohydrates, nucleic acids and the like are known to those of skill in the art. In certain aspects, a targeting moiety can be a non-targeting moiety that is cross-linked or otherwise modified to bind one or more factors that associate with, modify or otherwise interact with a nucleic acid sequence.

In certain exemplary embodiments, a targeting moiety, a retrievable moiety and/or polynucleotide has a detectable label bound thereto. As used herein, the term “detectable label” refers to a label that can be used to identify a target (e.g., a factor associated with a nucleic acid sequence of interest, a chromosome or a sub-chromosomal region). Typically, a detectable label is attached to the 3′- or 5′-end of a polynucleotide. Alternatively, a detectable label is attached to an internal portion of an oligonucleotide. Detectable labels may vary widely in size and compositions; the following references provide guidance for selecting oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like.

Methods for incorporating detectable labels into nucleic acid probes are well known. Typically, detectable labels (e.g., as hapten- or fluorochrome-conjugated deoxyribonucleotides) are incorporated into a nucleic acid, such as a nucleic acid probe during a polymerization or amplification step, e.g., by PCR, nick translation, random primer labeling, terminal transferase tailing (e.g., one or more labels can be added after cleavage of the primer sequence), and others (see Ausubel et al., 1997, Current Protocols In Molecular Biology, Greene Publishing and Wiley-Interscience, New York).

In certain aspects, a suitable targeting moiety, retrievable moiety or detectable label includes, but is not limited to, a capture moiety such as a hydrophobic compound, an oligonucleotide, an antibody or fragment of an antibody, a protein, a peptide, a chemical cross-linker, an intercalator, a molecular cage (e.g., within a cage or other structure, e.g., protein cages, fullerene cages, zeolite cages, photon cages, and the like), or one or more elements of a capture pair, e.g., biotin-avidin, biotin-streptavidin, NHS-ester and the like, a thioether linkage, static charge interactions, van der Waals forces and the like (See, e.g., Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160). In certain aspects, a suitable targeting label, retrievable label or detectable label is an enzyme (e.g., a methylase and/or a cleaving enzyme). In one aspect, an antibody specific against the enzyme can be used to retrieve or detect the enzyme and accordingly, retrieve or detect an oligonucleotide sequence or factor attached to the enzyme. In another aspect, an antibody specific against the enzyme can be used to retrieve or detect the enzyme and, after stringent washes, retrieve or detect a factor or first oligonucleotide sequence that is hybridized to a second oligonucleotide sequence having the enzyme attached thereto.

Biotin, or a derivative thereof, may be used as an oligonucleotide label (e.g., as a targeting moiety, retrievable moiety and/or a detectable label), and subsequently bound by a avidin/streptavidin derivative (e.g., detectably labelled, e.g., phycoerythrin-conjugated streptavidin), or an anti-biotin antibody (e.g., a detectably labelled antibody). Digoxigenin may be incorporated as a label and subsequently bound by a detectably labelled anti-digoxigenin antibody (e.g., a detectably labelled antibody, e.g., fluoresceinated anti-digoxigenin). An aminoallyl-dUTP residue may be incorporated into an oligonucleotide and subsequently coupled to an N-hydroxy succinimide (NHS) derivatized fluorescent dye. In general, any member of a conjugate pair may be incorporated into a retrievable moiety and/or a detectable label provided that a detectably labelled conjugate partner can be bound to permit detection. As used herein, the term antibody refers to an antibody molecule of any class, or any sub-fragment thereof, such as an Fab.

Other suitable labels (targeting moieties, retrievable moieties and/or detectable labels) include, but are not limited to, fluorescein (FAM), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for reaction, retrieval and/or detection: biotin/-biotin, digoxigenin/a-digoxigenin, dinitrophenol (DNP)/-DNP, 5-Carboxyfluorescein (FAM)/-FAM.

Additional suitable labels (targeting moieties, retrievable moieties and/or detectable labels) include, but are not limited to, chemical cross-linking agents. Cross-linking agents typically contain at least two reactive groups that are reactive towards numerous groups, including, but not limited to, sulfhydryls and amines, and create chemical covalent bonds between two or more molecules. Functional groups that can be targeted with cross-linking agents include, but are not limited to, primary amines, carboxyls, sulfhydryls, carbohydrates and carboxylic acids. Protein molecules have many of these functional groups and therefore proteins and peptides can be readily conjugated using cross-linking agents. Cross-linking agents are well known in the art and are commercially available (Thermo Scientific (Rockford, Ill.)).

A detectable moiety, label or reporter can be used to detect a nucleic acid or nucleic acid probe as described herein. Oligonucleotide probes or nucleic acid probes described herein can be labeled in a variety of ways, including the direct or indirect attachment of a detectable moiety such as a fluorescent moiety, hapten, colorimetric moiety and the like. A location where a label may be attached is referred to herein as a label addition site or detectable moiety addition site and may include a nucleotide to which the label is capable of being attached. One of skill in the art can consult references directed to labeling DNA. Examples of detectable moieties include various radioactive moieties, enzymes, prosthetic groups, fluorescent markers, luminescent markers, bioluminescent markers, metal particles, protein-protein binding pairs, protein-antibody binding pairs and the like. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C, or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labeling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Alternatively, the above fluorophores and those mentioned herein may be added during oligonucleotide synthesis using for example phosphoroamidite or NHS chemistry. Protocols are known in the art for custom synthesis of nucleotides having other fluorophores (See, Henegariu et al. (2000) Nature Biotechnol. 18:345). 2-Aminopurine is a fluorescent base that can be incorporated directly in the oligonucleotide sequence during its synthesis. Nucleic acid could also be stained, a priori, with an intercalating dye such as DAPI, YOYO-1, ethidium bromide, cyanine dyes (e.g. SYBR Green) and the like.

Other fluorophores available for post-synthetic attachment include, but are not limited to, ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red (available from Molecular Probes, Inc., Eugene, Oreg.), Cy2, Cy3, Cy3.5, Cy5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver or gold particles may be used to enhance signal from fluorescently labeled nucleotide and/or oligonucleotide sequences (Lakowicz et al. (2003) BioTechniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Biotin/avidin is an example of a ligand-ligand binding pair. An antibody/antigen binding pair may also be used with methods described herein. Other ligand-ligand binding pairs or conjugate binding pairs are well known to those of skill in the art. Digoxigenin may be incorporated as a label and subsequently bound by a detectably labeled anti-digoxigenin antibody (e.g. fluoresceinated anti-digoxigenin). An aminoallyl-dUTP or aminohexylacrylamide-dCTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM, FITC), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

According to certain aspects, detectable moieties described herein are spectrally resolvable.

“Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

In certain embodiments, the detectable moieties can provide higher detectability when used with an electron microscope, compared with common nucleic acids. Moieties with higher detectability are often in the group of metals and organometals, such as mercuric acetate, platinum dimethylsulfoxide, several metal-bipyridyl complexes (e.g. osmium-bipy, ruthenium-bipy, platinum-bipy). While some of these moieties can readily stain nucleic acids specifically, linkers can also be used to attach these moieties to a nucleic acid. Such linkers added to nucleotides during synthesis are acrydite- and a thiol-modified entities, amine reactive groups, and azide and alkyne groups for performing click chemistry. Some nucleic acid analogs are also more detectable such as gamma-adenosine-thiotriphosphate, iododeoxycytidine-triphosphate, and metallonucleosides in general (see Dale et al., Proc. Nat. Acad. Sci. USA, Vol. 70, No. 8, pp. 2238-2242 (1973)). The modified nucleotides are added during synthesis. Synthesis may refer by example to solid support synthesis of oligonucleotides. In this case, modified nucleic acids, which can be a nucleic acid analog, or a nucleic acid modified with a detectable moiety, or with an attachment chemistry linker, are added one after each other to the nucleic acid fragments being formed on the solid support, with synthesis by phosphoramidite being the most popular method. Synthesis may also refer to the process performed by a polymerase while it synthesizes the complementary strands of a nucleic acid template. Certain DNA polymerases are capable of using and incorporating nucleic acids analogs, or modified nucleic acids, either modified with a detectable moiety or an attachment chemistry linker to the complementary nucleic acid template.

Detection method(s) used will depend on the particular detectable labels used in the reactive labels, retrievable labels and/or detectable labels. In certain exemplary embodiments, target nucleic acids such as chromosomes and sub-chromosomal regions of chromosomes during various phases of the cell cycle including, but not limited to, interphase, preprophase, prophase, prometaphase, metaphase, anaphase, telophase and cytokinesis, having one or more reactive labels, retrievable labels, or detectable labels bound thereto by way of the probes described herein may be selected for and/or screened for using a microscope, a spectrophotometer, a tube luminometer or plate luminometer, x-ray film, a scintillator, a fluorescence activated cell sorting (FACS) apparatus, a microfluidics apparatus or the like.

When fluorescently labeled targeting moieties, retrievable moieties, or detectable labels are used, fluorescence photomicroscopy can be used to detect and record the results of in situ hybridization using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

In certain exemplary embodiments, images of fluorescently labeled chromosomes are detected and recorded using a computerized imaging system such as the Applied Imaging Corporation CytoVision System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al., supra.

In situ hybridization methods using probes described herein can be performed on a variety of biological or clinical samples, in cells that are in any (or all) stage(s) of the cell cycle (e.g., mitosis, meiosis, interphase, G0, G1, S and/or G2). Examples include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. Samples are prepared for assays of the invention using conventional techniques, which typically depend on the source from which a sample or specimen is taken. These examples are not to be construed as limiting the sample types applicable to the methods and/or compositions described herein.

In certain exemplary embodiments, probes include multiple chromosome-specific probes, which are differentially labeled (i.e., at least two of the chromosome-specific probes are differently labeled). Various approaches to multi-color chromosome painting have been described in the art and can be adapted to the present invention following the guidance provided herein. Examples of such differential labeling (“multicolor FISH”) include those described by Schrock et al. (1996) Science 273:494, and Speicher et al. (1996) Nature Genet. 12:368). Schrock et al. describes a spectral imaging method, in which epifluorescence filter sets and computer software is used to detect and discriminate between multiple differently labeled DNA probes hybridized simultaneously to a target chromosome set. Speicher et al. describes using different combinations of 5 fluorochromes to label each of the human chromosomes (or chromosome arms) in a 27-color FISH termed “combinatorial multifluor FISH”). Other suitable methods may also be used (see, e.g., Ried et al., 1992, Proc. Natl. Acad. Sci. USA 89:1388-92).

Hybridization of the labeled probes described herein to target chromosomes sequences can be accomplished by standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides (e.g., hybridized Oligopaints). The reagents used in each of these steps and their conditions of use vary depending on the particular situation and whether their use is required with any particular probes. Hybridization conditions are also described in U.S. Pat. No. 5,447,841. It will be appreciated that numerous variations of in situ hybridization protocols and conditions are known and may be used in conjunction with the present invention by practitioners following the guidance provided herein.

### Example X

### REFERENCES

Each reference is incorporated herein by reference in its entirety for all purposes.
1000 Genomes Project Consortium, Durbin, R. M., Abecasis, G. R., Altshuler, D. L., Auton, A., Brooks, L. D., et al. (2010). A map of human genome variation from population-scale sequencing. Nature, 467(7319), 1061-1073. doi:10.1038/nature09534
Abyzov, A., Mariani, J., Palejev, D., Zhang, Y., Haney, M. S., Tomasini, L., et al. (2012). Somatic copy number mosaicism in human skin revealed by induced pluripotent stem cells. Nature. doi:10.1038/nature11629
Ahituv, N., Zhu, Y., Visel, A., Holt, A., Afzal, V., Pennacchio, L. A., & Rubin, E. M. (2007). Deletion of ultraconserved elements yields viable mice. PLoS Biology, 5(9), e234. doi: 10.1371/journal.pbio.0050234
Bejerano, G., Pheasant, M., Makunin, I., Stephen, S., Kent, W. J., Mattick, J. S., & Haussler, D. (2004). Ultraconserved elements in the human genome. Science, 304(5675), 1321-1325. doi:10.1126/science.1098119
Beroukhim, R., Mermel, C. H., Porter, D., Wei, G., Raychaudhuri, S., Donovan, J., et al. (2010). The landscape of somatic copy-number alteration across human cancers. Nature, 463(7283), 899-905. doi:10.1038/nature08822
Braconi, C., Valeri, N., Kogure, T., Gasparini, P., Huang, N., Nuovo, G. J., et al. (2011). Expression and functional role of a transcribed noncoding RNA with an ultraconserved element in hepatocellular carcinoma. Proceedings of the National Academy of Sciences of the United States of America, 108(2), 786-791. doi:10.1073/pnas.1011098108
Bruder, C. E. G., Piotrowski, A., Gijsbers, A. A. C. J., Andersson, R., Erickson, S., Diaz de Stahl, T., et al. (2008). Phenotypically concordant and discordant monozygotic twins display different DNA copy-number-variation profiles. American Journal of Human Genetics, 82(3), 763-771. doi:10.1016/j.ajhg.2007.12.011
Bullinger, L., Krönke, J., Schön, C., Radtke, I., Urlbauer, K., Botzenhardt, U., et al. (2010). Identification of acquired copy number alterations and uniparental disomies in cytogenetically normal acute myeloid leukemia using high-resolution single-nucleotide polymorphism analysis. Leukemia: Official Journal of the Leukemia Society of America, Leukemia Research Fund, U.K, 24(2), 438-449. doi:10.1038/1eu.2009.263
Calin, G. A., Liu, C.-G., Ferracin, M., Hyslop, T., Spizzo, R., Sevignani, C., et al. (2007). Ultraconserved Regions Encoding ncRNAs Are Altered in Human Leukemias and Carcinomas. Cancer Cell, 12(3), 215-229. doi:10.1016/j.ccr.2007.07.027
Calin, G. A., Sevignani, C., Dumitru, C. D., Hyslop, T., Noch, E., Yendamuri, S., et al. (2004). Human microRNA genes are frequently located at fragile sites and genomic regions involved in cancers. Proceedings of the National Academy of Sciences of the United States of America, 101(9), 2999-3004. doi:10.1073/pnas.0307323101
Campbell, C. D., Sampas, N., Tsalenko, A., Sudmant, P. H., Kidd, J. M., Malig, M., et al. (2011). Population-genetic properties of differentiated human copy-number polymorphisms. American Journal of Human Genetics, 88(3), 317-332. doi:10.1016/j.ajhg.2011.02.004
Cancer Genome Atlas Network. (2012). Comprehensive molecular characterization of human colon and rectal cancer. Nature, 487(7407), 330-337. doi:10.1038/nature11252
Cancer Genome Atlas Research Network. (2011). Integrated genomic analyses of ovarian carcinoma. Nature, 474(7353), 609-615. doi:10.1038/nature10166
Chiang, C. W. K., Derti, A., Schwartz, D., Chou, M. F., Hirschhorn, J. N., & Wu, C. T. (2008). Ultraconserved Elements: Analyses of Dosage Sensitivity, Motifs and Boundaries. Genetics, 180(4), 2277-2293. doi:10.1534/genetics.108.096537
Conrad, D. F., Pinto, D., Redon, R., Feuk, L., Gokcumen, O., Zhang, Y., et al. (2010). Origins and functional impact of copy number variation in the human genome. Nature, 464(7289), 704-712. doi:10.1038/nature08516
Curtis, C., Shah, S. P., Chin, S.-F., Turashvili, G., Rueda, O. M., Dunning, M. J., et al. (2012). The genomic and transcriptomic architecture of 2,000 breast tumours reveals novel subgroups. Nature. Retrieved from http://www.nature.com.ezp-prod1.hul.harvard.edu/nature/journal/vaop/ncurrent/full/nature10983.html?WT.ec_id=NATURE-20120419
Derti, A., Roth, F. P., Church, G. M., & Wu, C.-T. (2006). Mammalian ultraconserved elements are strongly depleted among segmental duplications and copy number variants. Nat Genet, 38(10), 1216-1220. doi:10.1038/ng1888
Drmanac, R., Sparks, A. B., Callow, M. J., Halpern, A. L., Burns, N. L., Kermani, B. G., et al. (2010). Human genome sequencing using unchained base reads on self-assembling DNA nanoarrays. Science, 327(5961), 78-81. doi:10.1126/science.1181498
ENCODE Project Consortium, Bernstein, B. E., Birney, E., Dunham, I., Green, E. D., Gunter, C., & Snyder, M. (2012). An integrated encyclopedia of DNA elements in the human genome. Nature, 489(7414), 57-74. doi:10.1038/nature11247
Fisher, S., Grice, E. A., Vinton, R. M., Bessling, S. L., & McCallion, A. S. (2006). Conservation of RET regulatory function from human to zebrafish without sequence similarity. Science, 312(5771), 276-279. doi:10.1126/science.1124070
Forsberg, L. A., Rasi, C., Razzaghian, H. R., Pakalapati, G., Waite, L., Thilbeault, K. S., et al. (2012). Age-related somatic structural changes in the nuclear genome of human blood cells. American Journal of Human Genetics, 90(2), 217-228. doi:10.1016/j.ajhg.2011.12.009
Hussein, S. M., Batada, N. N., Vuoristo, S., Ching, R. W., Autio, R., Närvä, E., et al. (2011). Copy number variation and selection during reprogramming to pluripotency. Nature, 471(7336), 58-62. doi:10.1038/nature09871
Iafrate, A. J., Feuk, L., Rivera, M. N., Listewnik, M. L., Donahoe, P. K., Qi, Y., et al. (2004). Detection of large-scale variation in the human genome. Nat Genet, 36(9), 949-951. doi:10.1038/ng1416
Jacobs, K. B., Yeager, M., Zhou, W., Wacholder, S., Wang, Z., Rodriguez-Santiago, B., et al. (2012). Detectable clonal mosaicism and its relationship to aging and cancer. Nat Genet. doi:10.1038/ng.2270
Jaeger, S. A., Chan, E. T., Berger, M. F., Stottmann, R., Hughes, T. R., & Bulyk, M. L. (2010). Conservation and regulatory associations of a wide affinity range of mouse transcription factor binding sites. Genomics, 95(4), 185-195. doi:10.1016/j.ygeno.2010.01.002
Jakobsson, M., Scholz, S. W., Scheet, P., Gibbs, J. R., VanLiere, J. M., Fung, H.-C., et al. (2008). Genotype, haplotype and copy-number variation in worldwide human populations. Nature, 451(7181), 998-1003. doi:10.1038/nature06742
kConFab Investigators, Walker, L. C., Krause, L., Spurdle, A. B., & Waddell, N. (2012). Germline copy number variants are not associated with globally acquired copy number changes in familial breast tumours. Breast Cancer Research and Treatment. doi:10.1007/s10549-012-2024-6
Kozomara, A., & Griffiths-Jones, S. (2011). miRBase: integrating microRNA annotation and deep-sequencing data. Nucleic Acids Research, 39(Database issue), D152-7. doi:10.1093/nar/gkq1027
Kritsas, K., Wuest, S. E., Hupalo, D., Kern, A. D., Wicker, T., & Grossniklaus, U. (2012). Computational analysis and characterization of UCE-like elements (ULEs) in plant genomes. Genome Research. doi:10.1101/gr.129346.111
Lampe, X., Samad, O. A., Guiguen, A., Matis, C., Remacle, S., Picard, J. J., et al. (2008). An ultraconserved Hox-Pbx responsive element resides in the coding sequence of Hoxa2 and is active in rhombomere 4. Nucleic Acids Research, 36(10), 3214-3225. doi:10.1093/nar/gkn148
Laurent, L. C., Ulitsky, I., Slavin, I., Tran, H., Schork, A., Morey, R., et al. (2011). Dynamic changes in the copy number of pluripotency and cell proliferation genes in human ESCs and iPSCs during reprogramming and time in culture. Cell Stem Cell, 8(1), 106-118. doi:10.1016/j.stem.2010.12.003
Laurie, C. C., Laurie, C. A., Rice, K., Doheny, K. F., Zelnick, L. R., McHugh, C. P., et al. (2012). Detectable clonal mosaicism from birth to old age and its relationship to cancer. Nat Genet. doi:10.1038/ng.2271
Lujambio, A., Portela, A., Liz, J., Melo, S. A., Rossi, S., Spizzo, R., et al. (2010). CpG island hypermethylation-associated silencing of non-coding RNAs transcribed from ultraconserved regions in human cancer. Oncogene, 29(48), 6390-6401. doi:10.1038/onc.2010.361
Matsuzaki, H., Wang, P.-H., Hu, J., Rava, R., & Fu, G. K. (2009). High resolution discovery and confirmation of copy number variants in 90 Yoruba Nigerians. Genome Biology, 10(11), R125. doi:10.1186/gb-2009-10-11-r125
Mayshar, Y., Ben-David, U., Lavon, N., Biancotti, J.-C., Yakir, B., Clark, A. T., et al. (2010). Identification and classification of chromosomal aberrations in human induced pluripotent stem cells. Cell Stem Cell, 7(4), 521-531. doi:10.1016/j.stem.2010.07.017
McCarroll, S. A., Kuruvilla, F. G., Korn, J. M., Cawley, S., Nemesh, J., Wysoker, A., et al. (2008). Integrated detection and population-genetic analysis of SNPs and copy number variation. Nat Genet, 40(10), 1166-1174. doi:10.1038/ng.238
Meireles-Filho, A. C. A., & Stark, A. (2009). Comparative genomics of gene regulation-conservation and divergence of cis-regulatory information. Curr Opin Genet Dev, 19(6), 565-570. doi: 10.1016/j.gde.2009.10.006
Mermel, C. H., Schumacher, S. E., Hill, B., Meyerson, M. L., Beroukhim, R., & Getz, G. (2011). GISTIC2.0 facilitates sensitive and confident localization of the targets of focal somatic copy-number alteration in human cancers. Genome Biology, 12(4), R41. doi:10.1186/gb-2011-12-4-r41
Mestdagh, P., Fredlund, E., Pattyn, F., Rihani, A., Van Maerken, T., Vermeulen, J., et al. (2010). An integrative genomics screen uncovers ncRNA T-UCR functions in neuroblastoma tumours. Oncogene, 29(24), 3583-3592. doi:10.1038/onc.2010.106
Michaelson, J. J., Shi, Y., Gujral, M., Zheng, H., Malhotra, D., Jin, X., et al. (2012). Whole-genome sequencing in autism identifies hot spots for de novo germline mutation. Cell, 151(7), 1431-1442. doi:10.1016/j.cell.2012.11.019
Nik-Zainal, S., Alexandrov, L. B., Wedge, D. C., Van Loo, P., Greenman, C. D., Raine, K., et al. (2012). Mutational Processes Molding the Genomes of 21 Breast Cancers. Cell. doi:10.1016/j.cell.2012.04.024
O'Huallachain, M., Karczewski, K. J., Weissman, S. M., Urban, A. E., & Snyder, M. P. (2012). Extensive genetic variation in somatic human tissues. Proceedings of the National Academy of Sciences of the United States of America. doi:10.1073/pnas.1213736109
Pennacchio, L. A., Ahituv, N., Moses, A. M., Prabhakar, S., Nobrega, M. A., Shoukry, M., et al. (2006). In vivo enhancer analysis of human conserved non-coding sequences. Nature, 444(7118), 499-502. doi:10.1038/nature05295
Piotrowski, A., Bruder, C. E. G., Andersson, R., Diaz de Stahl, T., Menzel, U., Sandgren, J., et al. (2008). Somatic mosaicism for copy number variation in differentiated human tissues. Human Mutation, 29(9), 1118-1124. doi:10.1002/humu.20815
Poitras, L., Yu, M., Lesage-Pelletier, C., Macdonald, R. B., Gagné, J.-P., Hatch, G., et al. (2010). An SNP in an ultraconserved regulatory element affects Dlx5/Dlx6 regulation in the forebrain. Development (Cambridge, England), 137(18), 3089-3097. doi:10.1242/dev.051052
Quinlan, A. R., Boland, M. J., Leibowitz, M. L., Shumilina, S., Pehrson, S. M., Baldwin, K. K., & Hall, I. M. (2011). Genome sequencing of mouse induced pluripotent stem cells reveals retro element stability and infrequent DNA rearrangement during reprogramming Cell Stem Cell, 9(4), 366-373. doi:10.1016/j.stem.2011.07.018
Sana, J., Hankeova, S., Svoboda, M., Kiss, I., Vyzula, R., & Slaby, O. (2012). Expression Levels of Transcribed Ultraconserved Regions uc.73 and uc.388 Are Altered in Colorectal Cancer. Oncology, 82(2), 114-118. doi:10.1159/000336479
Scaruffi, P., Stigliani, S., Moretti, S., Coco, S., De Vecchi, C., Valdora, F., et al. (2009). Transcribed-ultra conserved region expression is associated with outcome in high-risk neuroblastoma. BMC Cancer, 9(1), 441. doi:10.1186/1471-2407-9-441
Shaikh, T. H., Gai, X., Perin, J. C., Glessner, J. T., Xie, H., Murphy, K., et al. (2009). High-resolution mapping and analysis of copy number variations in the human genome: a data resource for clinical and research applications. Genome Research, 19(9), 1682-1690. doi:10.1101/gr.083501.108
Taher, L., McGaughey, D. M., Maragh, S., Aneas, I., Bessling, S. L., Miller, W., et al. (2011). Genome-wide identification of conserved regulatory function in diverged sequences. Genome Research, 21(7), 1139-1149. doi:10.1101/gr.119016.110
Taylor, B. S., Barretina, J., Socci, N. D., Decarolis, P., Ladanyi, M., Meyerson, M., et al. (2008). Functional copy-number alterations in cancer. PloS One, 3(9), e3179. doi: 10.1371/journal.pone.0003179
Taylor, B. S., Schultz, N., Hieronymus, H., Gopalan, A., Xiao, Y., Carver, B. S., et al. (2010). Integrative genomic profiling of human prostate cancer. Cancer Cell, 18(1), 11-22. doi:10.1016/j.ccr.2010.05.026
The Cancer Genome Atlas Network, Genome sequencing centres: Washington University in St Louis, Koboldt, D. C., Fulton, R. S., McLellan, M. D., Schmidt, H., et al. (2012). Comprehensive molecular portraits of human breast tumours. Nature. doi:10.1038/nature11412
The Cancer Genome Atlas Research Network, (Participants are arranged by area of contribution and then by institution.), Genome sequencing centres: Broad Institute, Hammerman, P. S., Lawrence, M. S., Voet, D., et al. (2012). Comprehensive genomic characterization of squamous cell lung cancers. Nature. doi:10.1038/nature11404
Vavouri, T., & Lehner, B. (2009). Conserved noncoding elements and the evolution of animal body plans. BioEssays: News and Reviews in Molecular, Cellular and Developmental Biology, 31(7), 727-735. doi:10.1002/bies.200900014
Visel, A., Prabhakar, S., Akiyama, J. A., Shoukry, M., Lewis, K. D., Holt, A., et al. (2008). Ultraconservation identifies a small subset of extremely constrained developmental enhancers. Nat Genet, 40(2), 158-160. doi:10.1038/ng.2007.55
Walter, M. J., Payton, J. E., Ries, R. E., Shannon, W. D., Deshmukh, H., Zhao, Y., et al. (2009). Acquired copy number alterations in adult acute myeloid leukemia genomes. Proceedings of the National Academy of Sciences of the United States of America, 106(31), 12950-12955. doi:10.1073/pnas.0903091106
Weirauch, M. T., & Hughes, T. R. (2010). Conserved expression without conserved regulatory sequence: the more things change, the more they stay the same. Trends in Genetics: TIG, 26(2), 66-74. doi:10.1016/j.tig.2009.12.002
Woolfe, A., Goodson, M., Goode, D. K., Snell, P., McEwen, G. K., Vavouri, T., et al. (2005). Highly conserved non-coding sequences are associated with vertebrate development. PLoS Biology, 3(1), e7. doi:10.1371/journal.pbio.0030007

What is claimed is:

1. A method of diagnosing an individual with a disease comprising
obtaining a cell sample from the individual,
comparing a maternal ultra-conserved element and a corresponding paternal ultra-conserved element, and
diagnosing the individual with a disease when the maternal ultra-conserved element differs from the paternal ultra-conserved element.

2. A method of treating an individual for a disease related to copy number variation of an ultra-conserved element in one or more cells comprising
triggering recognition by the cell of the copy number variation of the ultra-conserved element leading to cell apoptosis or elimination from a population of cells.

3. A method of purging deleterious cells having copy number variation of an ultra-conserved element from an individual comprising
triggering recognition by the cell of the copy number variation of the ultra-conserved element leading to cell apoptosis or elimination from a population of cells.

4. A method of purging a cell having copy number variation of an ultra-conserved element from a population of cells comprising
triggering recognition by the cell of the copy number variation of the ultra-conserved element leading to cell apoptosis, cell loss of fitness to survive or elimination from a population of cells.

5. A method of using ultra-conserved sequences to monitor and clear the genome of a population of cells from one or more cells having copy number variation of an ultra-conserved element comprising
triggering recognition by the cell of the copy number variation of the ultra-conserved element leading to cell apoptosis or elimination from a population of cells.

6. A method of eliminating cells from an individual comprising
causing a cell to compare ultra-conserved elements from maternal DNA with ultra-conserved elements from paternal DNA, and
wherein the cell becomes not viable if the ultra-conserved elements from the maternal DNA differ in sequence or copy number from the ultra-conserved elements from the paternal DNA.

7. A method of detecting a target nucleic acid comprising
Hybridizing a mixture of nucleic acid probes bearing a common binding site to a target nucleic acid, such as DNA of a chromosome,
binding a common secondary label to the hybridized nucleic acid probes and
detecting the hybridized labeled probes.
